(19) United States
(12) Patent Application Publication
(10) Pub. No.: US 2012/0190899 A1
(43) Pub. Date: Jul. 26, 2012

(54) HYDROGENATION CATALYST COMPRISING NICKEL ON CARBON

(75) Inventor: Annemarie Elisa Wilhelmina Beers, Utrecht (NL)

(73) Assignee: BASF SE, Ludwigshafen (DE)

(21) Appl. No.: 13/355,016

(22) Filed: Jan. 20, 2012

Related U.S. Application Data
(60) Provisional application No. 61/435,360, filed on Jan. 24, 2011.

(51) Int. Cl. C07C 29/132; B01J 37/16; C07C 29/14; B01J 21/18

(52) U.S. Cl. 568/863; 502/185; 568/861

(57) ABSTRACT

The hydrogenation catalyst comprises from 1 to 50% by weight, based on the total catalyst, of nickel on a carbon support, wherein the hydrogenation catalyst does not comprise any rhenium. Coconut shell carbon is preferably used as support.

The invention relates to a hydrogenation catalyst comprising nickel on a carbon support, a process for producing the hydrogenation catalyst and its use for the hydrogenation of sorbitol to glycols or the hydrogenation of glucose to sorbitol.

The preparation of chemical starting materials from renewable sources is gaining ever greater importance. Thus, for example, glycols such as propylene glycol and ethylene glycol can be produced from maize, with starch firstly being obtained from the maize and subsequently being converted into glucose, then sorbitol and subsequently glycols such as propylene glycol and ethylene glycol. These are important starting materials in the preparation of polymer resins such as polyurethanes or for the preparation of polymer crosslinkers and other chemical compounds.

The hydrogenation of sorbitol to glycols such as ethylene glycol and propylene glycol is carried out at high temperatures and pressures and also high pH values in an aqueous medium. Inorganic supports customarily used for hydrogenation catalysts generally withstand these conditions for only a short time, if at all, so that such catalysts are unsuitable for the hydrogenation of sorbitol.

As an alternative, catalysts comprising nickel and rhenium on a carbon support have been proposed. U.S. Pat. No. 6,841,085 describes the hydrogenation of sugars such as sorbitol to predominantly ethylene glycol and propylene glycol using a catalyst having 2.5% by weight of nickel and 2.5% by weight of rhenium on a coconut carbon support. In the production of the catalyst, the support is firstly impregnated with metal salt solutions of the active metals, subsequently dried and reduced at 280° C. for 16 hours.

A similar process is described in U.S. Pat. No. 7,038,094, in which a catalyst comprising rhenium and nickel on a coconut carbon support is likewise used.

It is an object of the present invention to provide hydrogenation catalysts, in particular for the hydrogenation of sorbitol to glycols, which withstand high temperatures and pressures and an aqueous environment having high pH values and can be produced simply and inexpensively. They should display preferential selectivity to propylene glycol and optionally ethylene glycol.

The object is achieved according to the invention by a hydrogenation catalyst comprising from 1 to 50% by weight, based on the total catalyst, of nickel on a carbon support, wherein the hydrogenation catalyst does not comprise any rhenium.

It has been found according to the invention that hydrogenation catalysts comprising nickel but no rhenium as active metal on a carbon support are suitable for the hydrogenation of sorbitol to glycols.

The catalysts can be obtained in a simple manner since only impregnation with an active metal is necessary. In addition, they are significantly cheaper than known catalysts since they dispense with the use of costly rhenium and use inexpensive carbon supports.

The catalyst of the invention does not contain any rhenium. This means that no technically effective amounts of rhenium are comprised in the catalyst and rhenium thus has no importance as active metal.

Preference is given to catalysts according to the invention which comprise only nickel as active metal. However, it is also possible for further active metals such as molybdenum, vanadium or tin or mixtures thereof to be present in addition to nickel.

The catalyst of the invention comprises nickel in an amount of from 1 to 50% by weight, preferably from 5 to 40% by weight, in particular from 10 to 30% by weight, based on the total catalyst. The proportion of further metals is from 0 to 25% by weight, preferably from 0 to 15% by weight, in particular from 0 to 5% by weight. If such metals are present, their minimum amount is preferably 0.5% by weight. Particular preference is given to no further active metals apart from nickel, iron, molybdenum, vanadium and/or tin being present on the catalyst support. Particular preference is given to only nickel being present as active metal on the catalyst support. In particular, the carbon support is impregnated only with nickel as metal.

According to the invention, any suitable carbon supports can be used. For example, it is possible to use coconut shells, olive stones or peat charcoal as carbon support. It is also possible to use synthetic carbon supports. Particular preference is given to using coconut shell carbon as support.

The carbon support can be used in untreated form or pretreated form for producing the catalyst. Pretreatment of the carbon can be carried out, for example, by means of heat, steam, acids or chemical pretreatment. A steam pretreatment of the coconut shell carbon with water vapor is particularly preferably carried out.

The support can have any suitable particle size. The support preferably has an average particle diameter in the range from 0.5 to 5 mm, particularly preferably from 0.75 to 3.5 mm, in particular from 1 to 2 mm.

The hydrogenation catalyst used according to the invention can be produced by any suitable processes. It is usually produced by impregnation of the support with a nickel salt solution, subsequent drying and subsequent reduction. The reduction is preferably carried out at a temperature above 300° C., particularly preferably in the range from >300° C. to 700° C., in particular in the range from 400 to 600° C., especially in the range from 400 to 500° C. For example, the reduction treatment can be carried out at about 500° C.

An increased reduction temperature leads to more active catalysts which allow a higher sorbitol conversion. Particularly good results are obtained at a hydrogenation temperature of 500° C. However, the selectivity of the catalyst is not reduced by the increased activity. The reduction can be followed by stabilization in air, preferably at room temperature. The invention also provides a catalyst which can be produced by the above process.

The invention also provides a process for producing the above catalyst by impregnation of the carbon support with a nickel salt solution, subsequent drying of the impregnated support and subsequent reduction of the dried support at a temperature above 300° C. The abovementioned reduction temperatures are preferably employed here.

Impregnation can be carried out by any suitable impregnation methods. Preference is given to carrying out vacuum impregnation. Any suitable nickel salts can be employed here. Preference is given to using nickel nitrate as aqueous solution.

Drying is preferably carried out at a temperature in the range from 50 to 150° C. and atmospheric pressure or preferably under reduced pressure. Drying is particularly preferably carried out under vacuum or reduced pressure.

The reduction is preferably carried out in the presence of a gas comprising free hydrogen, in particular in a hydrogen atmosphere.

The reduction can be followed by stabilization of the nickel-comprising catalyst, for example in air at room temperature, in order to obtain a storable, stable nickel catalyst.

Vacuum impregnation leads to a very well dispersed nickel catalyst comprising nickel crystallites having an average crystallite size in the range from 2 to 15 nm. Very uniform crystallites which do not undergo appreciable agglomeration, if any, and do not form relatively large clusters even after prolonged use of the catalyst in the hydrogenation of sorbitol are present here.

Typical hydrogenation conditions in the hydrogenation of sorbitol are a temperature in the range from 150 to 350° C., preferably from 200 to 300° C., in particular about 250° C., a hydrogen pressure in the range from 50 to 300 bar, in particular about 150 bar, a sorbitol concentration of from 10 to 40% by weight in water, in particular about 20% by weight in water, an initial pH in the range from 12 to 13, for example set by addition of KOH.

The hydrogenation can also be carried out under the reaction conditions as are described in U.S. Pat. No. 6,841,085 and U.S. Pat. No. 6,479,713.

To determine the effectiveness and strength of the catalysts, the sorbitol hydrogenation is generally carried out at a temperature of 250° C., a hydrogen pressure of 150 bar, a pH of from 12 to 13 on a 20% strength by weight aqueous sorbitol solution.

The degree of reduction of the sorbitol is preferably in the range from 50 to 99%.

After hydrogenation for a period of about 300 minutes, the strength of the catalyst is determined. No reduction in the strength as a result of the hydrogenation is found for the carbon supports, in particular the coconut shell carbon supports.

The catalysts of the invention are thus preferably used for the hydrogenation of sorbitol to glycols, in particular propylene glycol and ethylene glycol, with small amounts of glycerol, or for the hydrogenation of glucose to sorbitol.

The invention therefore also provides a process for preparing glycols by hydrogenation of sorbitol, in which the hydrogenation is carried out over a catalyst as described above.

In addition, the invention provides a process for preparing sorbitol by hydrogenation of glucose, wherein the hydrogenation is carried out over a catalyst as described above.

The hydrogenation is preferably carried out continuously, with the hydrogenation catalyst being present as a fixed bed.

The hydrogenation of sorbitol gives propylene glycol as main product, and also ethylene glycol in a significantly smaller amount and even significantly smaller amounts of glycerol. Xylitol, butanediol and methanol and also lactic acid are typically formed as by-products.

The formation of methane, as occurs in the case of the known catalysts, does not occur to an appreciable extent according to the invention.

Compared to known catalysts, the catalysts of the invention display improved selectivity in respect of the preparation of propylene glycol. In particular, the selectivity is very high in the case of nickel catalysts having coconut shell carbon supports.

Conversion and selectivity to propylene glycol and ethylene glycol are significantly better in the case of the nickel-comprising hydrogenation catalyst of the invention than in the case of a comparative catalyst which additionally comprises rhenium. Both the conversion and the propylene glycol selectivity were significantly better in the case of a catalyst comprising 10% by weight of nickel on a carbon support than in the case of a catalyst comprising 10% by weight of nickel and 1% by weight of rhenium on the same carbon support.

In the process for the hydrogenation of glucose for preparing sorbitol, the reaction is preferably carried out at a temperature in the range from 50 to 250° C., particularly preferably from 90 to 140° C., a pressure in the range from 30 to 250 bar, particularly preferably from 60 to 150 bar, and a glucose concentration in the preferably aqueous glucose solution in the range from 30 to 70% by weight, particularly preferably from 40 to 60% by weight. In a continuous process, the space velocity is preferably from 0.15 to 2 l/l·h.

An addition of base is typically not necessary. After about 300 hours, the strength of the catalyst in the fixed bed had not changed.

Compared to known catalysts, the catalysts of the invention display improved selectivity and activity in respect of the preparation of sorbitol. In particular, the selectivity is very high in the case of nickel catalysts and coconut shell carbon supports.

The invention is illustrated by the following example.

### EXAMPLE 1

### Production of the Catalyst

Carbon extrudates or granulated carbon were used as starting materials. However, coconut shell carbon as can be obtained from Japan EnviroChemicals Ltd. under the trade name SHIRASAGI C2X8/12 was preferably used. This carbon has a bulk density of about 0.5 g/ml and an average particle size of 1.8 mm.

An aqueous solution comprising nickel nitrate in deionized water having, for example, a nickel concentration of 14.4% by weight was firstly produced. For example, 53.3 g of Ni(NO₃)_{2.6} H₂O in 22.0 g of water was used for the impregnation of 50 g of carbon extrudates.

A vacuum impregnation was carried out as impregnation. The carbon support was maintained under reduced pressure for 30 minutes, after which it was spray impregnated with the above solution comprising nickel nitrate. Heating and drying of the impregnated support followed. The vacuum was then broken and air was allowed to flow in.

To reduce the impregnated catalyst support, this was heated to a reduction temperature of 410° C. or 450° C. or 500° C. at a heating rate of 60° C./h under nitrogen (100 ml/h). Hydrogen was then slowly introduced. After addition of 100% of the hydrogen, the catalyst was maintained at this temperature for 4 hours. It was then quickly cooled to 50° C. under nitrogen (100 ml/h). Air was then slowly admitted in order to carry out stabilization of the catalyst.

1. A hydrogenation catalyst comprising from 1 to 50% by weight, based on the total catalyst, of nickel on a carbon support, wherein the hydrogenation catalyst does not comprise any rhenium.

2. The hydrogenation catalyst according to claim 1, wherein coconut shell carbon is used as support.

3. The hydrogenation catalyst according to claim 1, wherein the carbon support is pretreated by means of heat, steam, acids or chemically.

4. The hydrogenation catalyst according to claim 1, wherein the support has an average particle diameter in the range from 0.5 to 5 mm.

5. The hydrogenation catalyst according to claim 1, wherein the support has been impregnated with a nickel salt solution, then dried and reduced at a temperature above 300° C.

6. A process for producing a hydrogenation catalyst according to claim 1 by impregnation of the carbon support with a nickel salt solution, subsequent drying of the impregnated support and subsequent reduction of the dried support at a temperature above 300° C.

7. The process according to claim 6, wherein the reduction is carried out at a temperature of from 400 to 600° C.

8. The process according to claim 6, wherein the support is pretreated by means of heat, steam, acids or chemically before impregnation.

9. A process for preparing glycols by hydrogenation of sorbitol, wherein the hydrogenation is carried out over a catalyst according to claim 1.

10. A process for preparing sorbitol by hydrogenation of glucose, wherein the hydrogenation is carried out over a catalyst according to claim 1.

11. The process according to claim 9, wherein the hydrogenation is carried out continuously and the hydrogenation catalyst is present as a fixed bed.
